(19) United States
(12) Patent Application Publication
(10) Pub. No.: US 2023/0301520 A1
(43) Pub. Date: Sep. 28, 2023

(54) SPECTROSCOPY SYSTEM FOR IDENTIFYING LIGHT SOURCE

(71) Applicant: GYRUS ACMI, INC. D/B/A OLYMPUS SURGICAL TECHNOLOGIES AMERICA, Westborough, MA (US)

(72) Inventors: Sergey A. Bukesov, Acton, MA (US); Kurt G. Shelton, Bedford, MA (US); Maria Rao, Worcester, MA (US); David Marcus, Northborough, MA (US)

(21) Appl. No.: 18/183,532

(22) Filed: Mar. 14, 2023

Related U.S. Application Data
(60) Provisional application No. 63/383,627, filed on Nov. 14, 2022.
(60) Provisional application No. 63/269,939, filed on Mar. 25, 2022.

(51) Int. Cl. A61B 5/00; G01N 21/31

(52) U.S. Cl. CPC A61B 5/0075; G01N 21/314; A61B 1/0684

(57) ABSTRACT

A method of identifying an illumination light source in a surgical system comprises steps of receiving a signal from a target after the target is illuminated by the illumination light source, performing spectroscopic analysis of the received signal, and based at least in part on the spectroscopic analysis, determining a characteristic of the illumination light source. A method of treating a target comprises receiving a signal from the target after the target is illuminated by an illumination light source, performing spectroscopic analysis of the received signal, based at least in part on the spectroscopic analysis, determining a first characteristic of the illumination light source and a second characteristic of the target, and based at least in part on the determined characteristics of the illumination light source and the target, operating a surgical system to treat the target.

### PRIORITY CLAIM

This application claims the benefit of priority to U.S. Provisional Pat. Application Serial No. 63/269,939, filed Mar. 25, 2022, and U.S. Provisional Pat. Application Serial No. 63/383,627, filed Nov. 14, 2022; the contents of which are hereby incorporated by reference in their entireties.

### TECHNICAL FIELD

This document pertains generally, but not by way of limitation, to systems and methods for performing spectroscopy on a material. More specifically, but not by way of limitation, the present application relates to systems and methods of identifying a light source in an endoscopy system using a spectroscopy system.

### BACKGROUND

Many surgical procedures involve the treatment or removal of target tissue, e.g., diseased, potentially diseased, or otherwise unwanted tissue, located inside of a patient. As such, some of these procedures require access to the internal anatomy of the patient via an open procedure or through a smaller opening in minimally invasive (e.g., endoscopic or laparoscopic) procedures.

It can be useful to positively identify the type or composition of tissue that is being removed or treated from the anatomy before it is removed or treated to, among other things, thereby ensure that the correct tissue is being removed or treated. For example, it can be useful to distinguish between healthy tissue and diseased tissue, such as cancerous tissue, to facilitate removal of the diseased tissue and not the healthy tissue.

### OVERVIEW

The present inventors have recognized, among other things, that problems to be solved in performing spectroscopic identification of tissue is the different spectroscopic response of different types of light sources to the tissue. A typical surgical instrument, such as an endoscope, can include various light delivery systems for projecting different types of light at a distal end of the instrument. For example, different types of light can be used for illumination, aiming and treatment. Illumination systems can use different types of light sources, such as Xenon, light emitting diodes (LED), halogen and laser diodes (LD). Furthermore, lasers used in medical instruments for illumination or treatment can use different laser technologies, such as holmium:yttrium-aluminum-garnet (Ho:YAG) and thulium-fiber (Tm-Fiber).

The medical instrument typically only includes light conductors that extend along the length of the device and a light emitter that projects the light out of the medical instrument. However, the light source that generates the light, whether illumination or treatment, can be located outside of the medical instrument, such as in an external computing system mounted on a stand or tower. Hospitals can have generators that produce different types of laser light and illumination light for the same instrument. As such, it is possible for different light sources to be used with the same medical instrument.

The present inventors have recognized that it can be difficult to identify a characteristic (e.g., a type, a composition, etc.) of the target tissue using a spectroscopic analysis without identifying the characteristic (e.g., type) of the light generator, which can accommodate a variety of different types of light for the same or different tissue types. In particular, spectral analysis of tissue is analyzed based on particular pairs of a light source and a target tissue. For example, target tissue is analyzed by comparing spectral analysis of a particular light source predetermined spectrographs for different target tissues. Thus, if a different light source is used, the spectral analysis of the tissue can be skewed or inaccurate.

The present subject matter can provide solutions to this problem and other problems, such as by providing medical devices, systems and methods that can identify the type of light being used for spectral analysis of the target tissue. The type of light can be identified before the spectral analysis of the tissue is performed. For example, spectral analysis of light from the light source reflected from a white surface or collected from ambient reflection can be performed to positively identify the illumination light source. Thereafter, the identity of the target tissue can be determined by analyzing light reflected from target tissue using spectral analysis using spectrographs of the correct light type to identify the target tissue. In examples, the light source for performing the spectroscopic analysis can be the illumination light source.

Results of the spectral analysis can be compared with library information for combinations of different tissue, different types of light sources (or generators). The library of information can optionally be accessed via the cloud and analyzed using artificial intelligence. The artificial intelligence analysis can be used to make or suggest adjustments to the light generation, for any or all of the treatment light source, spectroscopy light source and illumination light source, or surgical operation, such as the settings for generation/operation mode, power levels, shape and the like of the light source or surgical device (e.g., a laser system). Generation of the light and/or operation of the surgical device can then be adjusted, automatically, semiautomatically or manually, based on the artificial intelligence output to improve the ability of the light to positively identify the tissue type.

Spectroscopy techniques are widely used for identification of materials through the spectrum of light reflected, transmitted, emitted, or absorbed by the material. Examples of spectroscopy systems are described in Pub. No. US 2022/0039641, Pub. No. US 2021/0038300, Pub. No. US 2021/0038306, and Pub. No. US 2021/0038310, and Pub. No. US 2021/0038064.

In an example, a method of identifying an illumination light source in a surgical system comprises steps of receiving a signal from a target after the target is illuminated by the illumination light source, performing spectroscopic analysis of the received signal, and based at least in part on the spectroscopic analysis, determining a characteristic of the illumination light source.

In another example, a method of treating a target comprises receiving a signal from the target after the target is illuminated by an illumination light source, performing spectroscopic analysis of the received signal, based at least in part on the spectroscopic analysis, determining a first characteristic of the illumination light source and a second characteristic of the target, and based at least in part on the determined characteristics of the illumination light source and the target, operating a surgical system to treat the target.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a schematic illustration of a surgical system having a surgical instrument connected to a laser system, an imaging system including an illumination light source and a spectroscopy system, which can be connected via the Internet of Things (IOT) to the cloud and an artificial intelligence (AI) input.

FIG. 2 is a graph showing light intensity versus wavelength plots for different light sources.

FIG. 3 is a schematic diagram showing an exemplary computer-based clinical decision support system (CDSS) that is configured to provide operating parameters for light systems of a medical device system based on reflected or emitted spectroscopic signals.

FIG. 4 illustrates examples of feedback-controlled laser treatment systems.

FIG. 5 is a block diagram illustrating operations in methods of identifying and adjusting light sources in spectroscopic surgical systems.

FIG. 6 is a block diagram illustrating an example machine upon which any one or more of the techniques (e.g., methodologies) described herein can be performed.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

The present disclosure relates to identification of illumination light sources that illuminate an anatomical target during surgical procedures that can be performed using endoscopes, laparoscopes and the like. The illumination light can additionally be used to identify anatomic targets (e.g., target tissue) using spectroscopy. In additional examples, other types of light sources can be identified, such as light sources that can be used to perform treatment or intervention and light sources that can be used to perform spectroscopic analysis. It is desirable to identify all light sources, but particularly those used to perform spectroscopic analysis of anatomic targets, because spectroscopic analysis can depend on correlating the reflected spectroscopic signal to data sets of spectrographs for known light sources. As mentioned, hospitals can use different light sources with the same surgical instrument, thereby introducing a potential variable into the spectrographic analysis. The present disclosure provides methods of identifying a light source type using a first spectroscopy analysis, with the light source being subsequently used to identify a target tissue using a second spectroscopy procedure. For example, a spectroscopy system can be first used to identify an illumination light source, such as a Xenon light, an LED light a halogen light source or a laser diode, etc., so that the correct spectrographs can be used to analyze the spectroscopic signals generated by the target tissue. Thereafter, treatment of the anatomic target can be performed with a different light source, such as a laser. Identification of the illumination light source can be performed with or without the laser source emitting light. These methods can be part of algorithms and operations to identify the light source and the anatomical target and, in response, control and adjust composition, formation or emission of light (spectroscopic, treatment and illumination light) used in the surgical procedure, or control and adjust operation of a surgical device during the surgical procedure. As such, the controlled and adjusted light, or surgical device, can help prevent endoscope damage or tissue damage from improper or sub-optimal laser emission, detect broken light-emitting fibers and have other positive benefits, such as optimizing treatment effects at the anatomical target.

A light signal from the anatomical target can be rapidly detected and delivered by a delivery system to the spectroscopy system though, for example, a laser fiber or separate fiber channel. The delivery and spectroscopy systems can continuously collect spectral data from the target, deliver the signal to the spectrometer, and send the digital spectral data from the spectrometer to the feedback analyzer.

The feedback analyzer can analyze the spectroscopic signal data and compare it with available database libraries. The feedback analyzer can identify the illumination light source type (or treatment light source type) and/or characteristic(s) of the target based on the data analyses. The anatomical target identification helps optimize operation setup of the laser modules, preferable laser operation mode (pulse or continuous wave (CW)), power and energy, pulse shape and profile, laser emission pulse regimes and combines all of the generated pulses into a combined output pulse train. The optimized signal with the suggested settings will be send directly to the laser controller (automatic mode) or request operator approval to auto adjust the laser controller settings (semi-automatic mode).

In the present invention, the Internet of Things (IoT) system is a network where the components of the laser system can communicate and interact with the others over the Internet. In examples, spectral data can be delivered over IoT for subsequent analysis. Data can also be delivered to the laser system over IoT. This data can include, without being limited, configuration parameters, software update files, messages for users of the laser system and so on. In examples where the spectra database library is at least partly accessible through an IoT connection, the laser system can communicate with a remotely stored spectra database library to provide data for the feedback analyzer. In addition, all the components of the laser system can be remotely monitored and controlled if desired through the network.

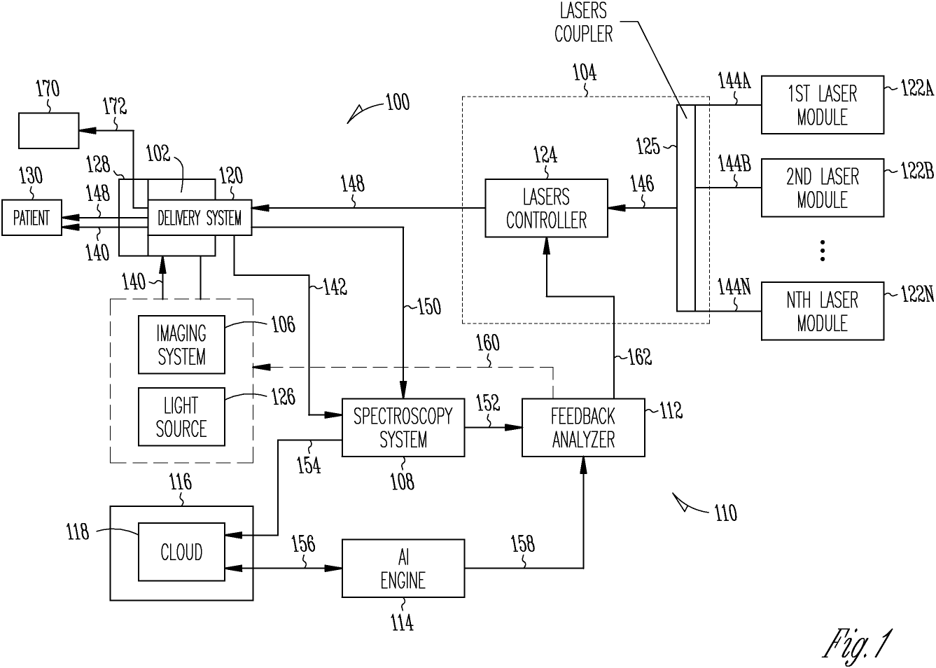

FIG. 1 is a schematic illustration of surgical system 100 having surgical instrument 102 connected to laser system 104, imaging system 106, spectroscopy system 108, and feedback control system 110. Feedback control system 110 can comprise feedback analyzer 112 and artificial intelligence (AI) engine 114. Spectroscopy system 108 and AI engine 114 can be connected via the Internet of Things (IOT) 116 to the cloud 118.

Surgical instrument 102 can be coupled to delivery system 120. Surgical instrument 102 can comprise an endoscope and delivery system 120 can comprise a light-emitting device, such as a laser-emitting lithotripsy device and/or an optical component (e.g., an optical fiber) associated with the light-emitting device for transmitting light to the target.

Laser system 104 can comprise any number of laser modules, such as laser module 122A, laser module 122B up to laser module 122N. Laser module 122A and laser module 122B can be connected to laser controller 124, such as via laser coupling system 125. Again, output from the laser system 104 can be transmitted to the target via the delivery system 120 (e.g., an optical fiber).

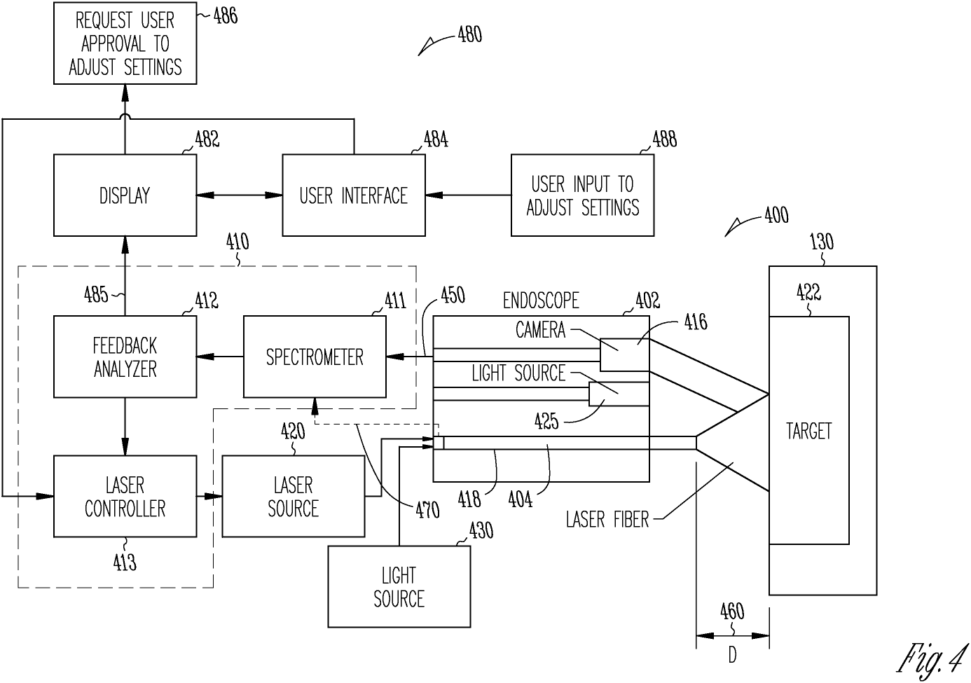

Imaging system 106 can be connected to light source 126 (“illumination light source”) and/or camera module 128. Camera module 128 can comprise a photosensitive element, such as a charge-coupled device (“CCD” sensor) or a complementary metal-oxide semiconductor (“CMOS”) sensor. Camera module 128 can be coupled (e.g., via wired or wireless connections) to imaging system 106 to transmit signals from the photosensitive element representing images (e.g., video signals) to imaging system 106, in turn to be displayed on a display such as an output unit or video monitor (e.g., display 482 of FIG. 4). In various examples, camera module 128 and imaging system 106 can be configured to provide outputs at desired resolution (e.g., at least 480p, at least 720p, at least 1080p, at least 4K UHD, etc.) suitable for endoscopy procedures.

Light source 126 can include an output port for transmitting light to surgical instrument 102, such as via a fiber optic link. Light source 126 can be configured to illuminate the anatomical region proximate the target tissue using light of desired spectrum (e.g., broadband white light, narrow-band imaging using preferred electromagnetic wavelengths, and the like). In examples, light source 126 can generate visible spectrum light using at least one Xenon generator or at least one Light Emitting Diode (LED).

An endoscope, e.g., surgical instrument 102, can be configured to be delivered through anatomy of a patient to reach a site of target anatomy to be treated or diagnosed. Delivery system 120 can be inserted into surgical instrument 102 (e.g., via a working channel thereof) to deliver treatment and diagnostic capabilities to the target anatomy. In the illustrated example, delivery system 120 includes an optical component that can be connected to camera module 128 to obtain video images of the target anatomy. In addition, visible light from light source 126 can additionally be delivered to the target anatomy through delivery system 120. The delivery system 120 may use the same or different optical components for delivering imaging data from the target anatomy to the camera module 128 and delivering visible light from the light source 126 to the target anatomy.

Laser system 104 can be used to deliver laser light to the target anatomy for various uses. Each of laser modules 122A - 122N can deliver a different type of laser light to laser controller 124. Laser controller 124 can coordinate delivery of the laser light from laser modules 122A - 122N with operation of appropriate controls on surgical instrument 102. Laser controller 124 can additionally be used to set the parameters of laser light emitted from laser modules 122A - laser module 122N, such as mode, power and shape.

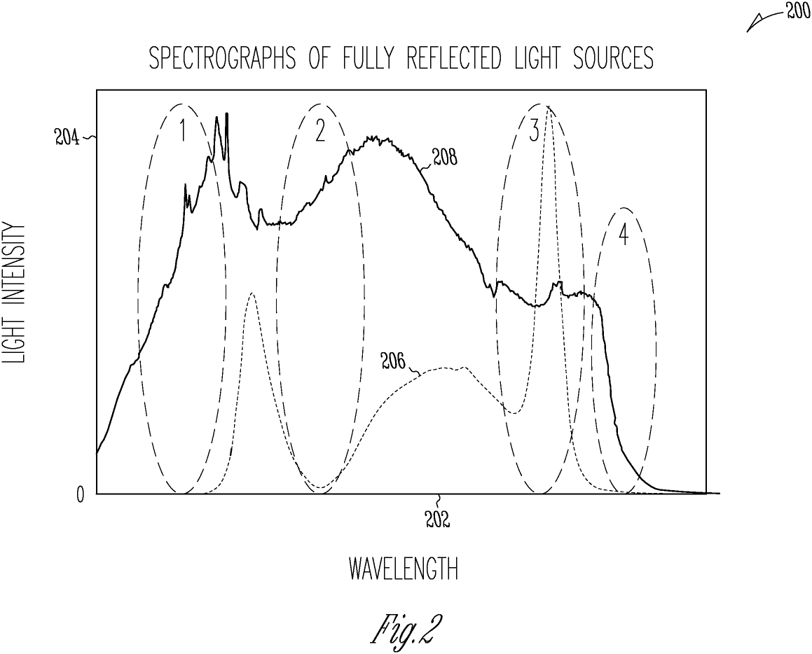

As discussed herein, light generated by Xenon and LED generators of light source 126 can both appear white to a user. Likewise, light emitted by laser modules 122A - 122N can be difficult to discern via the naked eye of the user. However, spectroscopic analysis from different light sources, even of the same type (e.g., illumination or laser) can have very different results. As such, it is important to identify the light generated by light source 126 and laser modules 122A - 122N, as well as any other light source used in an endoscope, to ensure that proper spectroscopic analysis of the light generated by the light source is performed. With the present disclosure, light from light source 126 and laser modules 122A - 122N can be analyzed to identify the type of light being emitted to thereby ensure that spectroscopy applied to such light is properly or optimally performed and to facilitate commensurate appropriate or optimal configurations for spectroscopy system 108, laser controller 124 and light source 126. In examples, positive identification of the light source is performed before spectrographic analysis is performed. Positive identification of the light source can be performed, for example, using spectrographs of various light sources showing the substantially full intensities (e.g., unabsorbed or fully reflected) of the light source across the wavelength spectrum, as shown in FIG. 2.

FIG. 2 shows an example of typical spectra for endoscope light sources that can be used for anatomical target identification. FIG. 2 illustrates graph 200 comprising x-axis 202 indicating wavelength in nanometers (nm) and y-axis 204 indicating light intensity in lumens per square meter (lux). Graph 200 shows plot 206 for a first type of illuminating light, such as from light source 126 and plot 208 for a second type of illuminating light, such as from light source 126. Thus, plots 206 and 208 can represent two different types of light source 126 or a light source that can switch between generating two different light types. In examples, plot 206 can comprise LED light and plot 208 can comprise Xenon light. Plot 206 and plot 208 indicate baseline spectrographs including the full spectrum and intensity of the light source without any light being absorbed by a reflecting surface. For example, plot 206 and plot 208 can be indicative of light reflected from a white surface or light directly emitted from a light source without reflection. Thus, plot 206 and plot 208 are not influenced by light absorption from tissue.

Plot 206 and plot 208 show the differences in light intensity for different wavelengths of different light sources. As can be seen, the waveforms for plot 206 and plot 208 are different, producing disparities in intensity across almost the entire range of wavelengths. There are several discrete locations where the intensity differential is particularly large, thereby providing discrepancies that can be readily recognized by feedback analyzer 112. For example, feedback analyzer 112 can compare numerical data representing actual reflected light spectroscopy to numerical data representing plot 206 and plot 208 to determine which plot the actual reflected light most closely resembles. In particular, graph 200 includes four different zones (Zone 1, Zone 2, Zone 3 and Zone 4) where the light source for plot 206 and the light source for plot 208 can be differentiated.

In examples, Zone 1 can be located proximate a wavelength of approximately 450 nm, Zone 2 can be located proximate a wavelength of approximately 525 nm, Zone 3 can be located proximate a wavelength of approximately 650 nm, and Zone 4 can be located proximate a wavelength of approximately 450 nm.

For Zone 1, Zone 2 and Zone 4, the intensity of plot 206 for a Xenon light source can be significantly, e.g., readily recognizable by machine interpretation, higher than the intensity of plot 208 for an LED light source. As such, +/- light intensity differential can be used to distinguish and identify the light sources.

For Zone 2 and Zone 3, the slope of plot 206 and plot 208 can be different. In particular, the slope of plot 206 can be decreasing or at a trough in Zone 2, while the slope of plot 208 in Zone 2 can be increasing, and the slope of plot 206 in Zone 3 can be increasing, while the slope of plot 208 in Zone 3 can be flat. As such, +/- slope differential, slope rise/decrease, and slope deflection points can be used to distinguish and identify the light sources.

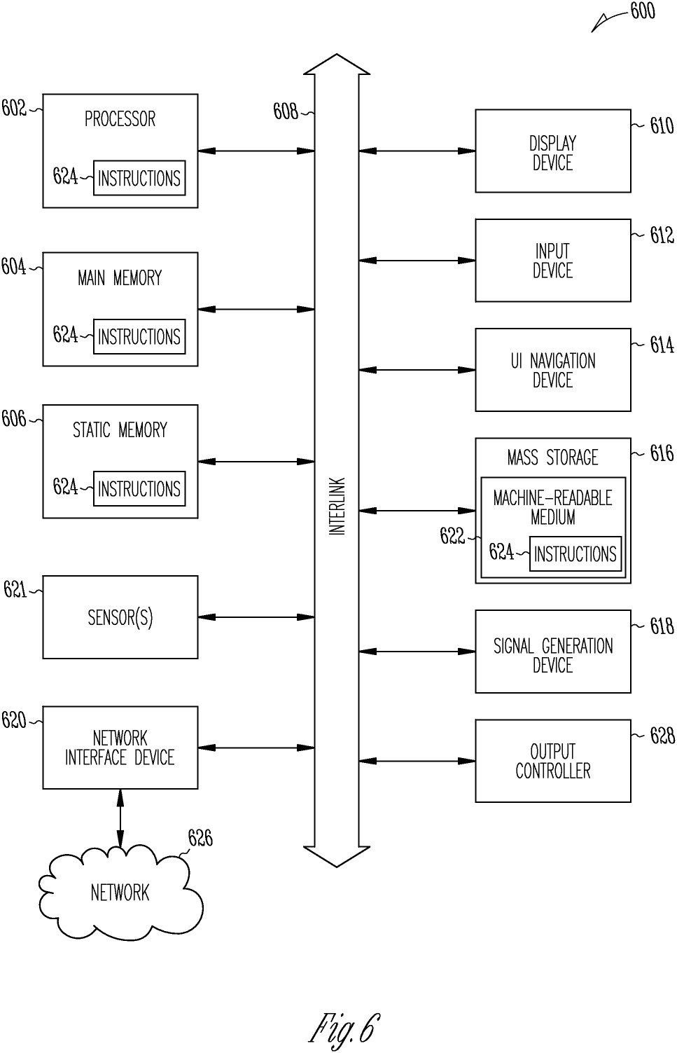

Spectroscopic analysis of the signal intensities and spectral slopes of plot 206 and plot 208 in Zone 1, Zone 2, Zone 3 and Zone 4 can allow for identification of the illumination light source type. Such information can be stored in memory of feedback analyzer 112 (e.g., memory 604 or memory 606 of FIG. 6) or in the cloud 118 for reference and comparison to waveforms collected by spectroscopy system 108 by feedback analyzer 112. In particular, numerical data sets for the formation of plot 206 and plot 208 can be stored in memory, such as memory 604 and memory 606 of FIG. 6, for comparison to data generated by spectroscopy system 108. Thus, spectroscopy system 108 can identify a light intensity value, or range of values, for light emitted from light source 126, and the magnitude of such identified light intensity values can be compared to values at the same wavelength from plot 206 and plot 208. In examples, feedback analyzer 112 can compare actual reflected light in Zone 1, Zone 2, Zone 3 and Zone 4 to look for common characteristics with plot 206 or plot 208.

Returning to FIG. 1, in operation, light source 126 can generate light beam 140 that can be passed to surgical instrument 102 via appropriate light conductors, such as fiber optic cables. Light beam 140 can be directed to patient 130. In particular, light beam 140 can be incident on anatomy within patient, such as anatomic target 422 of FIG. 4, via delivery system 120. Light beam 140 can be incident on anatomic target 422 and can then be reflected back to delivery system 120 as reflected illumination light beam 142. Additionally, light beam 140 can be incident on test target 170 and reflected back to delivery system 120 as reflected illumination light beam 142. As discussed herein, identification of light source 126 can be performed using reflected illumination light beam 142 and spectrographic analysis of anatomic target 422 can be performed using reflected illumination light beam 142.

Laser controller 124 can receive laser beams 144A, 144B - 144N from laser modules 122A, 122B - 122N, respectively. Laser beams 144A - 144N can be conveyed to laser controller 124 via laser coupling system 125. Laser controller 124 can receive combined laser beam 146. Laser controller 124 can perform various procedures to combined laser beam 146, such as by adjusting settings to control output powers, emission ranges, pulse shapes, and pulse trains, etc. Laser controller 124 can output treatment laser 148 to anatomic target 422. Treatment laser 148 can be directed to anatomic target 422 via delivery system 120. Treatment laser 148 can be incident on anatomic target 422 and can then be reflected back to delivery system 120 as reflected laser beam 150. In examples, spectrographic analysis of anatomic target 422 can be performed using reflected laser beam 150 and treatment of anatomic target 422 can be performed using treatment laser 148. Though not illustrated, treatment laser 148 can additionally be reflected off of test target 170 to facilitate identification of the source of treatment laser 148.

As discussed with reference to FIG. 4, spectroscopy system 108 can include spectrometer 411 and spectroscopy light source 430 (which may be the same or different from the endoscopic light source 126), which can additionally be incident on anatomic target 422 and reflected back to spectroscopy system 108. Though not illustrated, light from spectroscopy light source 430 can additionally be reflected off of test target 170 to facilitate identification of the type of light that spectroscopy light source 430 generates.

Spectroscopy system 108 can perform spectroscopic analysis on reflected illumination light beam 142 and reflected laser beam 150, as well as reflected light from light source 430, whether reflected from anatomic target 422 or test target 170. Spectroscopy system 108 can provide spectroscope signal 152 to feedback analyzer 112 and data signal 154 to IOT 116, which can communicate with the cloud 118. IOT 116 can provide signal 156 to AI engine 114. AI engine 114 can provide signal 158 to feedback analyzer 112. Feedback analyzer 112 can provide light signal 160 to light source 126 and laser signal 162 to laser controller 124.

In examples where light from light source 126 is used for the spectroscopic analysis of anatomic target 422 (FIG. 4) of patient 130, before a surgical procedure is performed, surgical system 100 can be operated to produce light beam 140 and reflected light beam 172. Light beam 140 can be reflected from test target 170. Test target 170 can comprise an object or surface that can reflect all or substantially all of the light of light beam 140 such that none of nearly none of light beam 140 is absorbed by test target 170. In examples, test target 170 can comprise surface of surgical system 100, such as a surface on a cabinet or housing of imaging system 106. In examples, reflected light beam 172 can simply be light of light beam 140 transmitted back through delivery system 120 without specifically being reflected from a target, e.g., portions of light beam 140 reflected from ambient light. Then, spectroscopy system 108 can analyze reflected light beam 172 so that feedback analyzer 112 can determine the type of light being generated by light source 126. For example, feedback analyzer 112 can, with or without the aid of AI engine 114, compare output of spectroscopy system 108 based on reflected light beam 172 to plots of the full spectrum of known light sources (e.g., FIG. 2) in order to identify light source 126. Spectroscopy of reflected light beam 172 can be performed by matching intensity values of reflected light beam 172 at various wavelengths to intensity and wavelength pairs from plots 206 and 208 of FIG. 2. If a match is found, feedback analyzer 112 can confirm that light source 126 is compatible with feedback analyzer 112, i.e., feedback analyzer 112 has access to spectrographs of different target tissues for the light type of light source 126. Thus, feedback analyzer 112 can be used in a subsequent step in conjunction with reflected illumination light beam 142 from patient 130 to provide an indication of the type of anatomy (e.g., the identity of anatomic target 422) that reflected illumination light beam 142 was incident on and reflected from. Thereafter, feedback analyzer 112 can provide recommendations for settings of laser controller 124 to carry out the surgical procedure, as well as potential adjustments for light source 126. However, if feedback analyzer 112 cannot find a match between the spectroscopic analysis of reflected light beam 172 and a baseline spectrograph of un-reflected light (e.g., FIG. 2), surgical system 100 can provide feedback to a user that an unknown illumination light source is being used. In other words, feedback analyzer 112 does not have access to, either in local memory or cloud 118, a combination of the used light source and spectroscopic analysis of that light type with anatomic target 422 and thus cannot provide confirmation of the target tissue type. In examples, surgical system 100 can shut down or disable surgical system 100 in full or in part if the illumination light source is not compatible with the tissue-identification capabilities of feedback analyzer 112. For example, surgical system 100 can only shut down tissue-identification capabilities of surgical system 100 such that a surgeon can proceed to use laser system 104 to perform the surgical procedure using surgeon skill to manually identify anatomic target 422. In such scenarios where feedback analyzer 112 cannot confirm the type of light source and anatomic target, surgical system 100 can still provide cloud 118 with the spectroscopic output so that AI engine 114 can learn new pairings of light sources and anatomic targets.

In FIG. 1, surgical system 100 is shown schematically according to various examples of the present disclosure. Further details of the construction and operation of surgical system 100 are discussed below, which is additionally applicable to laser treatment system (surgical system) 400 of FIG. 4 where indicated.

### Laser System 104

Surgical system 100 can include laser system 104 configured to deliver laser energy directed toward a target, and feedback control system 110 configured to be coupled to laser system 104. Laser system 104 can include one or more laser modules 122A - 122N (e.g., solid-state laser modules) that can emit similar or different wavelength from UV to IR. The number of the integrated laser modules, their output powers, emission ranges, pulse shapes, and pulse trains are selected to balance system costs and the performance required to deliver the desired effects to the targets. In examples, some or all of these factors, e.g., output powers, emission ranges, pulse shapes, and pulse trains, can be adjusted either by a user or automatically by laser controller 124 or feedback analyzer 112, to provide enhanced performance.

Laser modules 122A - 122N can be integrated with a fiber, and can be included in laser controller 124. Fiber-integrated laser systems can be used for endoscopic procedures due to their ability to pass laser energy through a flexible endoscope and to effectively treat hard and soft tissue. These laser systems produce a laser output beam in a wide wavelength range from UV to IR area (e.g., 200 nm to 10000 nm). Some fiber integrated lasers produce an output in a wavelength range that is highly absorbed by soft or hard tissue, for example 1900 – 3000 nm for water absorption or 400 - 520 nm for oxy-hemoglobin and/or deoxy-hemoglobin absorption. Various IR lasers can be used as the laser source in endoscopic procedures, such as those describe with referenced to Table 1.

TABLE 1

Examples of Light Sources for Laser Modules 122A -122N (FIG. 1 )

| Laser | Wavelength | Absorption Coefficient | Optical Penetration Depth |
|---|---|---|---|
| | λ (nm) | µ₀ (cm⁻¹) | δ (µm) |
| Thulium fiber laser: | 1908 | 88 / 150 | 114 / 67 |
| Thulium fiber laser | 1940 | 120 / 135 | 83 / 75 |
| Thulium: YAG: | 2010 | 62/60 | 161 / 167 |
| Holinium: YAG: | 2120 | 24 / 24 | 417 / 417 |
| Erbium:YAG: | 2940 | 12.000 / 1.000 | 1 / 10 |

Laser modules 122A - 122N can each consist of a number of solid-state laser diodes integrated into an optical fiber in order to increase output power and deliver the emission to the target. Some fiber integrated lasers can produce an output in a wavelength range that is minimally absorbed by the target soft or hard tissue. These types of lasers can provide effective tissue coagulation due to a penetration depth that similar to the diameter of a small capillary 5-10 µm. Laser modules 122A - 122N can comprise fiber-integrated laser modules and as described according to various examples in this disclosure have several advantages. In an example, the light emitting by one of laser modules 122A - 122N has a symmetric beam quality, circular and smooth (homogenized) intensity profile. The compact cooling arrangements is integrated into a laser module and make compact the whole system. Laser modules 122A - 122N can be easily combined with other fiber optic components. Additionally, fiber-integrated laser modules 122A - 122N can support standard optical fiber connectors that allow the modules to operate well with the most optical modules without alignment. Moreover, fiber-integrated laser modules 122A -122N can be easily replaced without changing the alignment of laser coupling system 125.

In some examples, one or more of laser modules 122A - 122N can produce a laser output in wavelength range that is highly absorbed by some materials such as soft or hard tissue, stone, bone, tooth etc., for example 1900 – 3000 nm for water absorption or 400 - 520 nm for oxy-hemoglobin and/or deoxy-hemoglobin absorption. In some examples one or more of laser modules 122A - 122N can produce a laser output in a wavelength range that is low absorbed by the target, such as soft or hard tissue, stone, bone, tooth etc. These types of lasers can provide more effective tissue coagulation due to a penetration depth that similar to the diameter of a small capillary (e.g., 5 - 10 µm). Commercially available solid-state lasers are potential emitting sources for the laser modules. Examples of laser sources for laser modules 122A – 122N can include UV-VIS emitting InXGa1-XN semiconductor lasers, such as GaN (emission 515 - 520 nm) or InXGa1-XN (emission 370 - 493 nm), GaXAl1-XAs laser (emission 750 - 850 nm), or InXGal-Xas laser (emission 904 - 1065 nm). Such laser sources can also be applicable to tissue coagulation applications.

Feedback control system 110 can comprise one or more subsystems including, for example, spectroscopy system 108, feedback analyzer 112, and laser controller 124.

### Spectroscopy System 108

Spectroscopy system 108 can include spectrometer 411 (FIG. 4) that can be used to analyze light from various sources, such as light source 126 used for laser modules 122A -122N used for treatment, as well as light source 430 (FIG. 4) included for spectroscopy system 108 used for spectroscopic purposes.

Spectroscopy system 108 can send a control light signal from light source 430 to a target, such as, but not limited to, a calculi, soft or hard tissue, bone, or tooth, or industrial targets, and collects spectral response data reflected from the target. The response can be delivered to spectrometer 411 through a separate fiber, laser fiber, or endoscope system, e.g., surgical instrument 102 (FIG. 1). Spectrometer 411 can send the digital spectral data to feedback analyzer 112. Examples of light sources for the spectroscopic system that cover an optical range from UV to IR can include those described above with reference to Table 2.

TABLE 2

Examples of Light Sources for Light Source 430 (FIG. 4 )

| | | |
|---|---|---|
| Application | Wavelength Range | Type |
| Color / VIS / NIR | 360-2500 nm | Tungsten Halogen |
| DUV | 190-400 nm | Deuterium |
| UV | 215-400 nm | Deuterium |
| UV/VIS/NIR reflection/absorption | 215-2500 nm | Deuterium/Halogen |
| UV/VIS/NIR absorption | 200-2500 nm | Deuterium/Halogen |
| UV/VIS | 200-1000 nm | Xenon |
| FTIR | 2000-25000 nm | Silicon Carbide |
| UV/VIS/IR Fluorescence | Multiple narrow emitting | LED, Laser Diode |

Spectroscopy system 108 can additionally be used to perform spectroscopic analysis of light from light source 126. Surgical instrument 102 can include suitable fibers for delivery of light from light source 126. Examples of light sources suitable for use as light source 126 are listed in Table 3.

TABLE 3

Examples of Light Sources for Light Source 126 (FIG. 1 )

| | | |
|---|---|---|
| Application | Wavelength Range | Type |
| High Power | 200-1000 nm | Xenon |
| Precise Wavelength | Multiple narrow emitting | LED |

Spectroscopy system 108 can additionally be used to perform spectroscopic analysis of light from laser modules 122A - 122N, such as those listed in Table 1.

Optical spectroscopy is a powerful method that can be used for easy and rapid analysis of organic and inorganic materials. Any light used for spectroscopic analysis can be integrated into a separate fiber channel, a laser fiber or an endoscope system. A light source signal reflected from the target can be rapidly collected and delivered to spectrometer 411 by an imaging system 106 containing a detector such as a CCD or CMOS sensor for example, which can be included in a digital endoscope. Other imaging systems like laser scanning can also be used for collecting spectroscopic response. The optical spectroscopy has several advantages. It can be easily integrated with a laser fiber in delivery system 120. It is a nondestructive technique to detect and analyze material chemical composition, and the analysis can be performed in real time. The optical spectroscopy can be used to analyze different types of materials including, for example, hard and soft tissue, calculi structures, etc.

Various spectroscopic techniques can be used alone or in combination to analyze target chemical composition and create the spectroscopic feedback. Examples of such spectroscopic techniques can include UV-VIS reflection spectroscopy, fluorescent spectroscopy, Fourier-Transform Infrared Spectroscopy (FTIR), or Raman spectroscopy, among others. Table 2 presents examples of light sources for the spectroscopy system 108 that cover an optical area from UV to IR and applicable to an example. Tungsten Halogen light sources are commonly used to do spectroscopic measurements in the visible and near IR range. Deuterium light sources are known for their stable output and they are used for UV absorption or reflection measurements. The mixes of the Halogen light with the Deuterium light produces a wide spectral range light source providing a smooth spectrum from 200-2500 nm. A Xenon light source is used in applications where a long lifetime and high output power is needed, such as in fluorescence measurements. LED and Laser Diodes light sources provide high power at a precise wavelength; they have long lifetime, short warm-up time and high-stability. A spectroscopy light source can be integrated into a separate fiber channel, laser fiber or endoscope system. A light source signal reflected from the target can be rapidly detected and delivered to the spectrometer though a separate fiber channel or laser fiber.

### Feedback Analyzer 112

Feedback analyzer 112 can receive inputs from various sources including spectroscopic response data from spectrometer 411 of spectroscopy system 108 and AI engine 114 to suggest or directly adjust laser system operating parameters, including those of laser modules 122A - 122N, or operating parameters of light source 126. In examples, feedback analyzer 112 can compare the spectroscopic response data, such as from beam 142, beam 150 and reflected light beam 172, to available database libraries of baseline spectrographs for various light sources 126, such as those of FIG. 2, and anatomic target composition data for various combinations of light source 126 or laser modules 122A - 122N and anatomic targets. Examples of tissue spectrographs for various types of light sources are described in Pub. No. US 2021/0038064 to Shelton et al., which is hereby incorporated by reference in its entirety. Based on the different spectroscopic system feedbacks, feedback analyzer 112 can detect light source 126 and composition of anatomic target 422, and suggest laser operating modes (also referred to as laser setups), such as operating parameters for at least one of laser modules 122A - 122N, to prevent damage of optical fibers, achieve effective tissue treatments for the identified tissue composition, and suggests an illumination light operating mode (also referred to as an illumination light setup), such as operating parameters for light source 126. Examples of the operating parameters for laser modules 122A - 122N that can be adjusted can include at least one laser wavelength, pulsed or continuous wave (CW) emission mode, peak pulse power, pulse energy, pulse rate, pulse shapes, and the simultaneous or sequenced emission of pulses from at least one laser module. Sequenced pulses include bursts of pulses which combine to deliver the selected pulse energy. Pulses as described herein refer generally to the time between starting and stopping a laser emission from a laser module. The intensity of the laser energy during each pulse can vary to have the shape of an increasing or decreasing ramp or sinusoidal profile, or any other shape alone or in combination with a sequence of pulses so long as the selected average laser power is maintained. For example, a 2 W average power setting with a pulse energy of 1 J occurs at a frequency of 2 Hz if there is only one pulse. However, the energy can also be delivered as two 0.5 J pulses in quick succession that occurs at a rate of 2 Hz. Each of those pulses can have similar pulse shapes, or different. Feedback analyzer 112 can utilize algorithms and input data to directly adjust or suggest laser operating parameters such as those described in the example above. Examples of the operating parameters for light source 126 that can be adjusted can include amplitude, brightness, power, wavelength and intensity. In examples, light intensity can be adjusted within a range of values depending on target material, application, and ambient light. Additionally, the wavelength range and spectra shape can be adjusted by additional optical filters. LED light source optical properties can also be adjusted by controlling the intensities of the constituent LEDs.

### Laser Controller 124

Laser controller 124 can be integrated with laser coupling system 125. Laser coupling system 125 can couple one or more of laser modules (e.g., solid-state laser modules) 122A - 122N into a fiber. Laser controller 124 can be coupled to feedback analyzer 112, which can send the optimized signal with the suggested settings directly to laser controller 124 (automatic mode), or request operator approval to adjust the laser settings (semi-automatic mode). FIG. 1 is a schematic diagram of a fully automated Laser System where laser controller 124 can be automatically adjusted by feedback analyzer 112. FIG. 4 is a schematic diagram of a semi-automated Laser System where surgical system 100 requires user approval, such as via a display 482 including user interface 484. In an example, the laser settings can be adjusted within a set range, which in an example, can be predetermined by the user at the start of the procedure.

In some examples, laser controller 124 can combine two or more laser pulse trains to create a combined laser pulse train. Laser controller 124 can generate a number of (e.g., N) laser pulse trains, combine the laser pulse trains into a combined pulse train, and expose the target with the combined pulse train. The different laser trains can be turned on at different times, and/or turned off at different times, in accordance with the feedback analyzer signal. The output combined laser pulse train can include portions where two or more of the laser trains overlap in time.

With the combination of laser modules 122A - 122N, spectroscopy system 108, feedback analyzer 112, and feedback control system 110 as described herein can continuously identify the composition of a target through an endoscope and update the laser settings throughout a procedure.

The main components of laser system 104 can be easily customized depending on the targeted medical procedure. For example, laser controller 124 can support different lasers types and their combination. This allows a wider range of output signal options including power, wavelength, pulse rates, pulse shape and profile, single laser pulse trains and combined lasers pulse trains. The operating mode of laser system 104 can be automatically adjusted, or suggested for each desired optical effect. Spectroscopy system 108 can collect information about the target materials that is useful for diagnostic purposes, and for confirming that laser parameters are optimal for the target. Feedback analyzer 112 can automatically optimize operation mode of the laser system and reduces risk of human mistake.

### Internet of Things (IoT) System 116

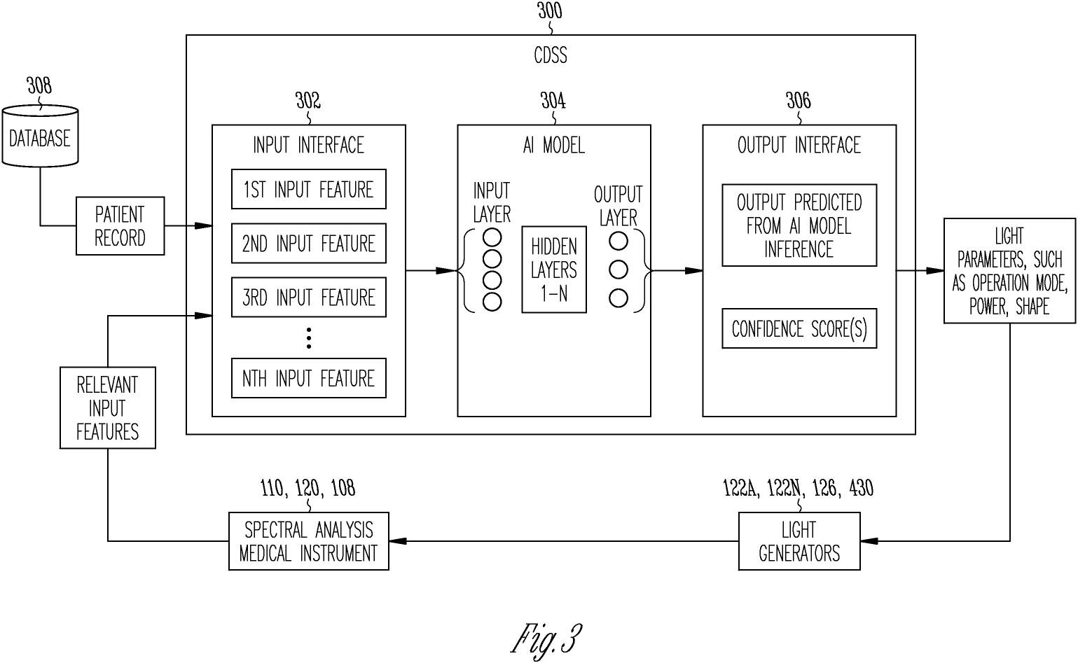

In some examples, surgical system 100 can include IoT system 116 that supports storing the spectral database library on cloud 118, supports quick access to the spectra and optimal setup database library, and enables communication between cloud 118 and feedback analyzer 112. The spectral database library can include 1) predetermined spectrographs for different combinations of light sources that can be used to identify the light source and 2) predetermined spectrographs for different combinations of light sources and anatomic tissues that can be used to identify anatomic targets. The cloud storage of data supports the use of artificial intelligence (AI) techniques to provide input to feedback analyzer 112, and supports immediate access to algorithm and database improvements, as is described in greater detail with reference to FIG. 3.

According to various examples described herein, IoT system 116 can include a network where the components of surgical system 100 can communicate and interact with others over the Internet. IoT supports quick access to the spectra database library stored on cloud 118 and performs communication between cloud 118 and feedback analyzer 112. In addition, all of the components of surgical system 100 can be remotely monitored and controlled if needed through the network. An example of such successful connection is the Internet of Medical Things (also called the Internet of Health Things) is an available application of the IoT for medical and health related purposes, which include data collection and analysis for research, and monitoring.

In various examples, IoT system 116 can support access to various cloud resources including cloud-based detection, recognition, or classification of spectroscopy light sources and a target structure (e.g., calculi structures or anatomical tissue). In some examples, a machine learning (ML) engine, such as AI model 304 of FIG. 3, can be implemented in cloud 118 to provide services of cloud-based target detection, identification, or classification. The ML engine can include a trained ML model (e.g., machine-readable instructions executable on one or more microprocessors).

The ML engine can receive spectroscopic data for spectroscopy light sources, e.g., light source 126 and light source 430, from laser system 104 or retrieve target spectroscope data stored in the cloud 118, perform light source detection, identification, or classification, and generate an output such as a label representing a light source type (e.g., Xenon or LED.

The ML engine can receive target spectroscopic data from laser system 104 or retrieve target spectroscope data stored in the cloud 118, perform target detection, identification, or classification, and generate an output such as a label representing a tissue type (e.g., normal tissue or cancerous lesion, or tissue at a particular anatomical site) or a calculus type (e.g., kidney, bladder, pancreobiliary, or gallbladder stone having a particular composition).

The light source and target spectroscopic data, among other clinical data collected from the patient before or during a procedure, can be automatically uploaded to cloud 118 at the end of the procedure or other scheduled time. Alternatively, a system user (e.g., a clinician) can be prompted to upload the data to cloud 118. In some examples, the output can additionally include a probability of the target being identified as tissue or calculi, or a probability of the target being classified as a particular tissue type or a calculus type. A system user (e.g., a clinician) can use such cloud services to obtain near real-time information about target tissue or calculi in vivo such as while performing an endoscopic laser procedure.

In some examples, the ML engine can include a training module configured to train a ML model using training data such as stored in cloud 118. The training data can include spectroscopic data associated with light source and target information, such as a tag identifying target types (e.g., calculi types, or tissue types). The training data can include lab data based on spectroscopic analysis of a variety of light sources and tissue types and/or calculi types. Additionally or alternatively, the training data can include clinical data acquired from multiple patients in vitro or in vivo. In some examples, patient-identifying information can be removed from the patient clinical data (e.g., spectroscopic data) prior to such data being used uploaded to cloud 118 to train the ML model or to perform target detection, identification, or classification using a trained ML model. System 100 can associate the de-identified patient clinical data with a tag identifying source of data (e.g., hospital, laser system identification, procedure time). The clinician can analyze and confirm target type (e.g., calculi or tissue type) during or after the procedure, and associate the light source and target type with the de-identified patient clinical data to form the training data. Using the de-identified patient clinical can advantageously increase the robustness of the cloud-based ML model as additional data from a large patient population can be included to train the ML model. This can also enhance the performance of the ML model to recognize rare calculi types as the spectroscopic data from rare calculi types are difficult to obtain clinically or from a lab.

Various ML model architectures and algorithms can be used, such as decision trees, neural networks, deep-learning networks, support vector machines, etc. In some examples, the training of the ML model can be performed continuously or periodically, or in near real time as additional spectroscopic data is made available. The training involves algorithmically adjusting one or more ML model parameters, until the ML model being trained satisfies a specified training convergence criterion. The resultant trained ML model can be used in cloud-based target detection, recognition, or classification. With a ML model trained by exploiting large volume of data stored in cloud 118 and additional data constantly or periodically added thereto, the ML based target recognition with cloud connection as described herein can improve the accuracy and robustness of in vivo target detection, recognition, and classification.

FIG. 3 shows a schematic diagram of an exemplary computer-based clinical decision support system (CDSS) 300 that is configured to identify light types and tissue types and generate light generation parameters to better identify and treat the tissue and improve performance of the system based on spectroscopy of light reflected from anatomic tissue, such as wavelength and light intensity CDSS 300 can comprise an example of AE Engine 114 of FIG. 1.

CDSS 300 can comprise input interface 302, AI Model 304, output interface 306, and can be connected to database 308. Input interface 302 can be connected to feedback control system 110 and can thus receive inputs from spectroscopy system 108, feedback analyzer 112 and delivery system 120, including reflected illumination light beam 142 and reflected laser beam 150.

In various embodiments, CDSS 300 includes input interface 302 through which spectral analysis or spectroscopic information, such as wavelength, light intensity and spectra shape, which are specific to a procedure for a patient, are provided as input features to artificial intelligence (AI) model 304. Additional inputs can comprise illumination light type, treatment light type, target tissue type and surgical procedure type. Additional other inputs can also be provided, such as whether AI model 304 is performing a light source identification procedure or a tissue identification procedure. A processor can perform an inference operation in which spectral analysis output is applied to the AI model to generate light parameters, and a user interface (UI) through which the light parameters are communicated to a user, e.g., a clinician.

In some embodiments, input interface 302 can be a direct data link, via wired or wireless or Internet or IoT system 116 (FIG. 1), between the CDSS 300 and one or more medical devices that generate at least some of the input features. Database 308 can reside on the cloud 118 (FIG. 1). Input interface 302 can transmit spectral analysis data directly to the CDSS 300 during a therapeutic and/or diagnostic medical procedure. Additionally, or alternatively, input interface 302 can be a classical user interface that facilitates interaction between a user and CDSS 300. For example, input interface 302 can facilitate a user interface through which the user can manually enter light parameters, such as mode, power and shape. Additionally, or alternatively, input interface 302 can provide CDSS 300 with access to an electronic patient record from which one or more input features can be extracted. In any of these cases, input interface 302 can be configured to collect one or more of the following input features in association with a specific patient on or before a time at which CDSS 300 is used to assess light type and tissue type:

In examples, the 1^{st} input feature can comprise a light type, such as illumination or laser.

In examples, the 2^{nd} input feature can comprise a specific light type, such as Xenon, LED, halogen, LD, etc.

In examples, the 3^{rd} input feature can comprise a specific light type, such as Ho:YAG, Tm-Fiber, etc.

In examples, the 4^{th} input feature can comprise a tissue type, such as healthy tissue, diseased tissue, etc.

In examples, the 5^{th} input feature can comprise an anatomic tissue type, such as kidney, uterus, intestine, stomach, etc.

In examples, the 6^{th} input feature can comprise a light wavelength.

In examples, the 7^{th} input feature can comprise a light intensity.

In examples, the 8^{th} input feature can comprise spectra shape, such as slope.

Other input features can additionally be used consistent with the present disclosure. Also, not all of the input features can be used.

Based on one or more of the above input features, the processor performs an inference operation using the AI model to generate the light parameters. For example, input interface 302 can deliver the light type and tissue, light intensity and wavelength, and spectra shape type into an input layer of the AI model which propagates these input features through the AI model to an output layer. The AI model can provide a computer system the ability to perform tasks, without explicitly being programmed, by making inferences based on patterns found in the analysis of data. AI model explores the study and construction of algorithms (e.g., machine-learning algorithms) that can learn from existing data and make predictions about new data. Such algorithms operate by building an AI model from example training data in order to make data-driven predictions or decisions expressed as outputs or assessments. In an example, AI model 304 can suggest a type of laser to be used to treat a particular type of tissue based on performance of laser and tissue combinations from previous surgical procedures stored in database 308. In another example, AI model 304 can suggest pulse trains for particular tissue and surgical procedures to better perform the surgery or more quickly perform the surgery based on results of previous surgical procedures stored in database 308. Results of the current surgical procedure being performed can thereafter be stored in database 308 such that AI model 304 can include additional datapoints for suggesting parameters of laser modules 122A - 122N. Thus, as database 308 grows, such as when surgeons utilize their own preferences over the suggested parameters of AI model 304, new surgical outcomes can be included that can facilitate AI model 304 adapting to suggesting different parameters for laser modules 122A - 122N.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled, and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model can be trained continuously or periodically prior to performance of the inference operation by the processor. Then, during the inference operation, the patient specific input features provided to the AI model can be propagated from an input layer, through one or more hidden layers, and ultimately to an output layer that corresponds to the light parameters. For example, spectroscopic wavelength and intensity can be provided to AI Model 304 from spectroscopy system 108. The wavelength and intensity can be used to identify a light source and/or a particular tissue, such as with feedback analyzer 112. AI Model 104 can analyze the spectroscopic signature of reflected light, e.g., wavelength and intensity, and identify the light source used to generate the wavelength and intensity. AI Model 104 can analyze the spectroscopic signature of reflected light, e.g., wavelength and intensity, and identify the anatomic tissue the light was reflected from. AI Model 104 can analyze the identified tissue for the given wavelength and intensity and can generate parameters for the identified light source and can change output parameters of laser modules 122A - 122N to improve the surgical procedure, such as laser operation mode (pulse or continuous wave (CW)), power and energy, pulse shape and profile, laser emission pulse regimes and combines all of the generated pulses into a combined output pulse train. The changed parameters generated by AI Model 304 can provide spectra shapes that can provide more definite indications that identify the tissue type, thereby improve the likelihood that the correct target tissue is the recipient of the surgical procedure. The changed parameters generated by AI Model 304 can provide laser parameters that are more effective at treating diseased tissue or that prevent damage to optical fibers, for example.

During and/or subsequent to the inference operation, the light parameters can be communicated to the user via the user interface (UI) and/or automatically cause the light generator to change the light generation parameters to those identified and suggested by AI Model 304. For example, the light generator can automatically change the light generation parameters, or input interface 302, via an output device such as a screen or display 482 (FIG. 4), can provide a prompt or message to the clinician with the suggested changes and ask the clinician to accept the changes using, for example, user interface 484 (FIG. 4). Furthermore, output interface 306 can output the tissue type so the clinician can verify the tissue upon which to perform the medical procedure. Additionally or alternatively, the medical device, such as an endoscope or a treatment device can automatically or with clinician guidance generate an ablation or ultrasound signal to treat the target tissue.

FIG. 4 illustrates an example feedback-controlled laser treatment system 400. In FIG. 4, laser treatment system 400 includes endoscope 402 integrated with feedback-controlled laser treatment system 400 that receives light source feedback. Laser treatment system 400, which is an example of surgical system 100 (FIG. 1), comprises endoscope 402, laser source 420, illumination light source 425 (e.g., light source 126) and spectroscic light source 430. In various examples, a portion or the entirety of the feedback-controlled laser treatment system 400 can be embedded into the endoscope 402. Feedback-controlled laser treatment system 400 can operate similarly as surgical system 100 of FIG. 1, with the addition of light source 430 that can be used to provide input to spectrometer 411, instead of in FIG. 1 spectroscopy system 108 using input from light source 126, and the addition of user-input system 480 that allows for user adjustment of laser controller 413, instead of feedback analyzer 112 directly adjusting laser controller 124 in FIG. 1. FIG. 4 additionally shows imaging signal 450 from endoscopic camera module 416 being provided to spectrometer 411.

Feedback-controlled laser treatment system 400 can include spectrometer 411, which can be included in spectroscopy system 108, feedback analyzer 412 (an example of at least a portion of feedback analyzer 112), and laser controller 413 (an example of laser controller 124). Laser source 420 can comprise an example of laser module 122A - 122N, and can be coupled to laser fiber 404. Fiber integrated laser systems can be used for endoscopic procedures due to their ability to pass laser energy through a flexible endoscope and to effectively treat hard and soft tissue. These laser systems produce a laser output beam in a wide wavelength range from UV to IR area (200 nm to 10000 nm). Some fiber integrated lasers produce an output in a wavelength range that is highly absorbed by soft or hard tissue, for example 1900 – 3000 nm for water absorption or 400 - 520 nm for oxy- hemoglobin and/or deoxy-hemoglobin absorption. Table 2 above is a summary of IR lasers that emit in the high water absorption range 1900 - 3000 nm and that are suitable for use as light source 430.

Some fiber integrated lasers produce an output in a wavelength range that is minimally absorbed by the target soft or hard tissue. These types of lasers provide effective tissue coagulation due to a penetration depth that similar to the diameter of a small capillary 5 -10 µm. Examples of laser source 420 can include UV-VIS emitting InxGa₁₋ₓN semiconductor lasers such as GaN laser with emission at 515 - 520 nm, InₓGa₁₋ₓN laser with emission at 370 -493 nm, GaxAl₁₋ₓAs laser with emission at 750 - 850 nm, or InₓGa₁₋ₓAs laser with emission at 904 - 1065 nm, among others.

Light source 430 can produce an electromagnetic radiation signal that can be transmitted to anatomic target 422 via a first optical pathway extending along the elongate body of endoscope 402. The first optical pathway can be located within working channel 418. In an example, the first optical pathway can be an optical fiber separate from laser fiber 404. In another example, the electromagnetic radiation signal can be transmitted through the same laser fiber 404 used for transmitting laser beams. The electromagnetic radiation exits the distal end of the first optical pathway and projects to the target structure and surrounding environment. Anatomic target 422 is within the view of endoscopic camera module 416 (e.g., camera module 128, such that in response to the electromagnetic radiation projecting to the target structure and surrounding environment, endoscopic camera module 416, such as a CCD or CMOS camera, can collect the signal reflected from anatomic target 422, produce an imaging signal 450 of the target structure, and deliver the imaging signal to the feedback-controlled laser treatment system 410. In some examples, imaging system other than the CCD or CMOS camera, such as laser scanning, can be used for collecting spectroscopic response.

In addition to or in lieu of the feedback signal (e.g., imaging signal 450) generated and transmitted through endoscopic camera module 416, in some examples, the signal reflected from the anatomic target 422 can additionally or alternatively be collected and transmitted to the feedback-controlled laser treatment system 410 through a separate fiber channel or a laser fiber such as associated with endoscope 402. In additional examples, laser treatment system 400 including endoscope 402 integrated with feedback-controlled laser treatment system 400 can be configured to receive spectroscopic sensor feedback. Reflected spectroscopic signal 470 (which can function similarly as reflected illumination light beam 142 and reflected laser beam 150 of FIG. 1) can travel back to feedback-controlled laser treatment system 410 through the same optical pathway, such as laser fiber 404, that is used for transmitting the electromagnetic radiation from light source 430 to anatomic target 422. In another example, reflected spectroscopic signal 470 can travel to the feedback-controlled laser treatment system 410 through a second optical pathway, such as a separate optical fiber channel from the first optical fiber transmitting the electromagnetic radiation from light source 430 to the target structure.

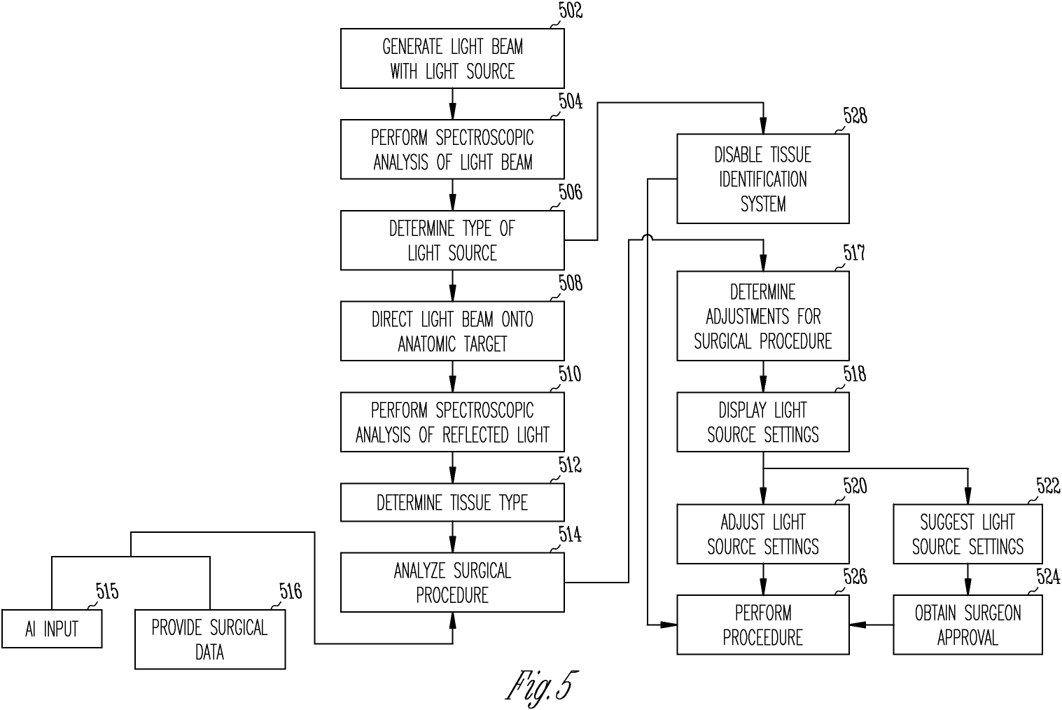

Feedback-controlled laser treatment system 400 can analyze one or more feedback signals (e.g., imaging signal 450 of the target structure or reflected spectroscopic signal 470 or spectroscope signal 152 or reflected illumination light beam 142 or reflected laser beam 150 of FIG. 1) to determine a light source, a tissue type and an operating state for one or more of laser source 420, illumination light source 425 and spectroscopy light source 430, such as by using the procedure outlined with reference to FIG. 5. Spectrometer 411 can generate one or more spectroscopic properties from the one or more feedback signals, such as by using one or more of a FTIR spectrometer, a Raman spectrometer, a UV-VIS spectrometer, a UV-VIS-IR spectrometer, or a fluorescent spectrometer. Feedback analyzer 412 can be configured to identify or classify the target structure as one of a plurality of structure categories or structure types, such as by using one or more of target detectors or target classifiers. Laser controller 413 can be configured to determine an operating mode of the laser source 420, illumination light source 425 and light source 430, as similarly discussed above with reference to FIG. 1.

FIG. 4 additionally shows feedback-controlled laser treatment system 400 comprising user-input system 480, which can comprise display 482 and user interface 484. User-input system 480 can receive signals from feedback analyzer 412 to provide output on display 482 including information relating to suggested changes for laser controller 413, illumination light source 425 and light source 430. In examples, settings for laser source 420 can be adjusted within a set range provided by display 482, which in an example can be predetermined by the user at the start of the procedure. Display 482 can receive signal 485 from feedback analyzer 412 indicative of recommended settings or ranges of settings for light source 430, illumination light source 425 and laser controller 413. Display 482 can display recommendation 486 to the user, including an audio signal or a visual or graphical representation of the recommended settings or ranges of settings for light source 430, illumination light source 425 and laser controller 413. The user can provide input 488 to affirm or deny the recommendation of signal 485 or select a specific setting from a recommended range of settings.

FIG. 5 is a block diagram illustrating operations in method 500 of identifying and adjusting light sources using spectroscopic surgical systems described herein. FIG. 5 illustrates an example of a sequence of operations that can be used in method 500. However, other steps consistent with the disclosure provided herein can be included. Additionally, some operations can be performed in a different order or omitted in additional examples.

At operation 502, a light beam can be generated with a light source. For example, light source 126 (FIG. 1) can generate light beam 140. Different types of light source 126 can be used with surgical system 100 (FIG. 1). For example, light source 126 can be configured to generate Xenon light or LED light. The light beam can be reflected from test target 170. Reflected light beam 172 can be received by spectroscopy system 108. For example, reflected light beam 172 can be received by delivery system 120 within surgical instrument 102 and passed through a light conductor, e.g., an optical fiber, to spectrometer 411 (FIG. 4). Surgical instrument 102 and delivery system 120 (FIG. 1) can be used to direct light beam at anatomic target 422 using, for example, optical fibers.

At operation 504, spectroscopy system 108 can perform a spectroscopic analysis of reflected light beam 172. For example, the intensity and wavelength of reflected light beam 172 can be compared to database information of intensities and wavelengths for different types of light sources. The light intensity can be compared to database information comprising light intensity values for different wavelengths of different types of light sources, such as different types of light source 126 that produce Xenon and LED light. In examples, spectroscopic information for the different types of light sources can be stored in spectroscopy system 108. In additional examples, spectroscopy system 108 can obtain spectroscopic information for the different types of light sources from cloud 118 (FIG. 1) or memory 604 (FIG. 6).

At operation 506, the intensity and wavelength data set that matches most closely with that of reflected light beam 172 can be used to identify the type of light coming from light source 126 or another light source. As such, spectroscopic analysis of light beam 140 for light source 126 can be used to verify the type of light source being used for spectroscopic tissue analysis to ensure that subsequent spectroscopic analysis of tissue will be properly executed. In examples, spectroscopic analysis of light beam 140 for light source 126 can be used to determine a manufacturer of light source 126.

Operations 502 - 506 are described with reference to determining the type of light emanating from light source 126. However, similar operations can be performed using light source 430 and laser modules 122A - 122N. That is, pure or undiminished light from light source 430 and laser modules 122A - 122N can be analyzed before being incident on target tissue.

At operation 508, a light beam can be generated with a light source, such as the same light beam that was generated at operation 502. For example, light source 126 (FIG. 1) can generate light beam 140. Light beam 140 can be reflected from target tissue upon which a medical procedure will be performed. For example, light beam 140 can be incident on anatomic target 422 of patient 130. Light beam 140 can reflect off anatomic target 422 as reflected illumination light beam 142. Surgical instrument 102 and delivery system 120 (FIG. 1) can be used to direct light beam at anatomic target 422 using, for example, optical fibers.

At operation 510, the reflected light from operation 504 can be received by a spectroscopy system. For example, reflected illumination light beam 142 can be received by delivery system 120 within surgical instrument 102 and passed through a light conductor, e.g., an optical fiber, to spectrometer 411. The received light can by analyzed by a spectroscope. For example, spectrometer 411 can analyze the light intensity for the wavelength of reflected illumination light beam 142. The light intensity can be compared to database information comprising light intensity values for different wavelengths of different types of anatomic tissue, such as calculi or cancer cells. In examples, spectroscopic information for the different types of anatomic targets can be stored in spectroscopy system 108. In additional examples, spectroscopy system 108 can obtain spectroscopic information for the different types of anatomic targets from cloud 118 (FIG. 1) or memory 604 (FIG. 6).

At operation 512, the type of anatomic tissue that reflected light beam 142 can be determined. For example, the light intensity for the wavelength of reflected illumination light beam 142 can be matched to a corresponding set of data points from predetermined reference data. Spectroscopy system 108 can, therefore, positively identify the type of tissue that produced the wavelength and light intensity combination. Thus, anatomic target 422 can be identified. As such, spectroscopic analysis of light beam 140 for anatomic target 422 can be used to verify the type tissue to ensure that the surgical procedure will be properly executed. In examples, method 500 can proceed directly to operation 526 from operation 512.

At operation 514, the surgical procedure being performed in conjunction with method 500 can be analyzed. For example, the surgical procedure can be analyzed to determine suggested settings, or ranges of settings, for components of surgical system 100 to perform the surgical procedure. The suggested settings can be used to improve the surgical outcome or facilitate easier performance of the surgical procedure. The surgical procedure can be analyzed using inputs from surgical system 100 at operation 516 and inputs from AI engine 114 at operation 515.

At operation 516, surgical data can be combined with the identified light source and the identified tissue type to analyze the surgical procedure. In examples, the surgical data can comprise the type of laser modules, e.g., laser modules 122A – 122N, being used in the surgical procedure, settings for laser modules 122A - 122N being used, the type of surgical instrument 102 being used, the type of delivery system 120 being used, etc. Furthermore, surgical system 100 can be initially configured to perform a particular type of procedure, such as to use one of more of laser modules 122A - 122N to ablate, cut or cauterize anatomic target 422 (FIG. 4), such as calculi or cancer cells. A surgeon can enter parameters into laser controller 124 to generate the type of treatment laser 148 (FIG. 1) desired to engage anatomic target 422, such as output powers, emission ranges, pulse shapes, and pulse trains. As such, inputs at operation 516 can comprise preferences of a surgeon based on their own past experiences or judgment.

At operation 515, AI inputs can be combined with the surgical data, identified tissue type and the identified light source type. AI engine 114 (FIG. 1) can include, or can be connected to a storage system, having a database, such as database 308, of information relating to multiple different types of surgical procedures that can be performed with one or both of light source 126 (FIG. 1) and laser modules 122A - 122N. In examples, cloud 118 can be connected to a server having memory (e.g., memory 604 or 606 of FIG. 6) with such information stored thereon. The information stored in cloud 118 or AI engine 114 can include combinations of parameters, including different types of light sources 126, different types of laser modules 122A - 122N, and different settings (e.g., output powers, emission ranges, pulse shapes, and pulse trains) for different types of surgical procedures (e.g., cancer removal, kidney stone removal, gallbladder stone removal, etc.). For example, for each type of procedure, the information can include outcome data (e.g., patient recovery, relapse, etc.) for different combinations of the surgical parameters.

At operation 517, adjustments for the surgical procedure being performed in conjunction with method 500 can be determined. For the light type from light source 126 identified in operation 510, AI engine 114 can recommend parameters that provide the best outcome for the patient. In examples, operation 517 can utilize AI model 304 to determine adjustments for the surgical procedure.

At operation 518, the determined adjustments for the surgical procedure of operation 517 can be displayed for user reference. In examples, spectroscopic analysis of light beam 140 for light source 126 can be used to suggest settings for light source 126, light source 430 and laser modules 122A - 122N. For example, display 482 (FIG. 4) can provide a visual or graphical output of a recommended setting for surgical system 100 or a range of values for a setting. The recommended settings can include, but are not limited to output powers, emission ranges, pulse shapes, and pulse trains for different types of laser modules 122A - 122N for different types of surgical procedures (e.g., cancer removal, kidney stone removal, gallbladder stone removal, etc.). Likewise, operating parameters for light source 126 that can be suggested can include amplitude, brightness, power, wavelength and intensity.

At operation 520, settings for the surgical procedure can be automatically adjusted. For example, feedback analyzer 112 can without user input, provide light signal 160 to light source 126 and laser signal 162 to laser controller 124 to apply the suggested changes.

At operation 522, adjustments for the surgical procedure can be presented to a user as suggested adjustments. For example, display 482 (FIG. 4) can provide a visual or graphical output of a recommended setting for surgical system 100 or a range of values for a setting. Display 482 can solicit Yes/No acceptance of various parameters or can solicit entry or selection of values within suggested ranges. Display 482 can also provide options for declining any or all of the suggested parameter changes such that a surgeon can utilize previously entered parameters.

At operation 524, surgeon approval for a recommended change or a surgeon selection from a recommended range of settings can be obtained. A user can interact with user interface 484 by providing a voice command or tactile input to select and/or confirm suggested settings presented at operation 522.

At operation 526, the surgical procedure can be performed. The surgical procedure can be performed using a correct or desirable combination of illumination light source, anatomic target and treatment light. The surgical procedure can be performed with parameters originally entered by a surgeon based on surgeon skill, preference and assessment, the surgical procedure can be performed with parameters determined by AI model 304, or a combination thereof.

At operation 528, tissue identification functionality can be shut down. In examples, spectroscopic analysis of light beam 140 and reflected light beam 172 for light source 126 can be used to disable a tissue verification system. For example, if operation 506 cannot determine the output of light source 126, the capability of surgical system to provide confirmation of tissue type can be disabled. Appropriate warnings can be provided on display 482. Thereafter, a surgeon can proceed to perform the surgical procedure using user-entered parameters for the system.

FIG. 6 illustrates generally a block diagram of an example machine 600 upon which any one or more of the techniques (e.g., methodologies or operations) discussed herein can be performed. Portions of this description can apply to the computing framework of various portions of the laser treatment system and spectroscopic analysis system in accordance with examples as discussed in this document.

In examples, machine 600 can operate as a standalone device or can be connected (e.g., networked) to other machines. In a networked deployment, machine 600 can operate in the capacity of a server machine, a client machine, or both in server-client network environments. In an example, machine 600 can act as a peer machine in peer-to-peer (P2P) (or other distributed) network environment. Machine 600 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, can include, or can operate by, logic or a number of components, or mechanisms. Circuit sets are a collection of circuits implemented in tangible entities that include hardware (e.g., simple circuits, gates, logic, etc.). Circuit set membership can be flexible over time and underlying hardware variability. Circuit sets include members that can, alone or in combination, perform specified operations when operating. In an example, hardware of the circuit set can be immutably designed to carry out a specific operation (e.g., hardwired). In an example, the hardware of the circuit set can include variably connected physical components (e.g., execution units, transistors, simple circuits, etc.) including a computer readable medium physically modified (e.g., magnetically, electrically, movable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components can be used in more than one member of more than one circuit set. For example, under operation, execution units can be used in a first circuit of a first circuit set at one point in time and reused by a second circuit in the first circuit set, or by a third circuit in a second circuit set at a different time.

Machine (e.g., computer system) 600 can include hardware processor 602 (e.g., a central processing unit (CPU), a graphics processing unit (GPU), a hardware processor core, or any combination thereof), main memory 604 and static memory 606, some or all of which can communicate with each other via an interlink (e.g., bus) 608. Machine 600 can further include display unit 610 (e.g., a raster display, vector display, holographic display, etc.), alphanumeric input device 612 (e.g., a keyboard), and user interface (UI) navigation device 614 (e.g., a mouse). In an example, display unit 610, input device 612 and UI navigation device 614 can be a touch screen display. Machine 600 can additionally include storage device (e.g., drive unit) 616, signal generation device 618 (e.g., a speaker), network interface device 620, and one or more sensors 621, such as a global positioning system (GPS) sensor, compass, accelerometer, or other sensors. Machine 600 can include output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Storage device 616 can include machine readable medium 622 on which is stored one or more sets of data structures or instructions 624 (e.g., software) embodying or utilized by any one or more of the techniques or functions described herein, such as the operations described with reference to FIG. 5. Instructions 624 can also reside, completely or at least partially, within main memory 604, within static memory 606, or within hardware processor 602 during execution thereof by machine 600. In an example, one or any combination of hardware processor 602, main memory 604, static memory 606, or the storage device 616 can constitute machine readable media.

While machine-readable medium 622 is illustrated as a single medium, the term “machine readable medium” can include a single medium or multiple media (e.g., a centralized or distributed database, and/or associated caches and servers) configured to store the one or more instructions 624.

The term “machine readable medium” can include any medium that is capable of storing, encoding, or carrying instructions for execution by machine 600 and that cause machine 600 to perform any one or more of the techniques of the present disclosure, or that is capable of storing, encoding or carrying data structures used by or associated with such instructions. Non-limiting machine-readable medium examples can include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media can include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

Instructions 624 can further be transmitted or received over communication network 626 using a transmission medium via network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, network interface device 620 can include one or more physical jacks (e.g., Ethernet, coaxial, or phone jacks) or one or more antennas to connect to communication network 626. In an example, network interface device 620 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by machine 600, and includes digital or analog communications signals or other intangible medium to facilitate communication of such software.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### EXAMPLES

Example 1 is a method of identifying an illumination light source in a surgical system, the method comprising steps of: receiving a signal from a target after the target is illuminated by the illumination light source; performing spectroscopic analysis of the received signal; and based at least in part on the spectroscopic analysis, determining a characteristic of the illumination light source.

In Example 2, the subject matter of Example 1 optionally includes comparing the spectroscopic analysis to database information including spectroscopic information for different light types and/or different materials of the target, wherein the determination of the characteristic of the illumination light source is based at least in part on the comparing of the spectroscopic analysis.

In Example 3, the subject matter of Example 2 optionally includes wherein the spectroscopic analysis comprises comparing at least one of an intensity or a spectrum of the received signal to intensities or spectra associated with various types of illumination light sources from the target located within the database information.

In Example 4, the subject matter of any one or more of Examples 2-3 optionally include wherein the target comprises a test target configured to reflect the illumination light without light absorption.

In Example 5, the subject matter of any one or more of Examples 2—4 optionally include wherein the illumination light source comprises at least one of a Xenon light source and an LED light source.

In Example 6, the subject matter of any one or more of Examples 2-5 optionally include retrieving database information from a server connected to the Internet.

In Example 7, the subject matter of Example 6 optionally includes wherein the database information comprises an artificial intelligence engine.

In Example 8, the subject matter of any one or more of Examples 1-7 optionally include adjusting at least one of a parameter or a characteristic of the illumination light source to improve spectroscopic analysis of tissue based on the illumination light source identified.

In Example 9, the subject matter of Example 8 optionally includes wherein the parameter includes at least one of a brightness, power, wavelength and intensity and the characteristic includes a type of the illumination light source.

In Example 10, the subject matter of any one or more of Examples 1-9 optionally include illuminating anatomic tissue with the illumination light; performing spectroscopic analysis of a reflection of the illumination light; and determining a characteristic of the anatomic tissue with the spectroscopic analysis.

In Example 11, the subject matter of Example 10 optionally includes wherein the characteristic includes one or more of a type, material, composition, a composition profile, a structure, and/or a hardness of the anatomic tissue.

In Example 12, the subject matter of any one or more of Examples 10-11 optionally include adjusting at least one of a parameter or a characteristic of a treatment light source to improve treatment of the anatomic tissue based on the type of the anatomic tissue.

Example 13 is a method of treating a target comprising: receiving a signal from the target after the target is illuminated by an illumination light source; performing spectroscopic analysis of the received signal; based at least in part on the spectroscopic analysis, determining a first characteristic of the illumination light source and a second characteristic of the target; and based at least in part on the determined characteristics of the illumination light source and the target, operating a surgical system to treat the target.

In Example 14, the subject matter of Example 13 optionally includes wherein operating the surgical system comprises generating or adjusting one or more parameters including an operation mode, a power or energy, a pulse shape profile, an emission pulse regime and/or a combined output pulse train of the surgical system.

In Example 15, the subject matter of Example 14 optionally includes wherein adjusting the one or more parameters is automatically performed by a controller.

In Example 16, the subject matter of any one or more of Examples 14-15 optionally include wherein adjusting the one or more parameters comprises prompting a user to accept suggested adjustments to the illumination light source.

In Example 17, the subject matter of any one or more of Examples 14-16 optionally include wherein at least one of the spectroscopic analysis, the parameter of the illumination light source or the characteristic of the illumination light source are communicated via the Internet.

In Example 18, the subject matter of Example 17 optionally includes utilizing an artificial intelligence engine to generate the one or more parameters.

In Example 19, the subject matter of any one or more of Examples 13-18 optionally include wherein receiving the signal from the target after the target is illuminated by the illumination light source comprises: receiving a first signal from a test target after the test target is illuminated by the illumination light source; and receiving a second signal from an anatomic target after the anatomic target is illuminated by the illumination light source.

In Example 20, the subject matter of Example 19 optionally includes wherein performing spectroscopic analysis of the received signal comprises: performing spectroscopic analysis of the first signal to identify a type of light emitted by the illumination light source; and performing spectroscopic analysis of the second signal to identify a characteristic of tissue of the anatomic target.

In Example 21, the subject matter of Example 20 optionally includes wherein the characteristic includes one or more of a type, material, composition, a composition profile, a structure, and/or a hardness of the tissue.

In Example 22, the subject matter of any one or more of Examples 20-21 optionally include wherein: the test target comprises a reflective surface; and the illumination light source comprises a Xenon light source or an LED light source.

Each of these non-limiting examples can stand on its own, or can be combined in various permutations or combinations with one or more of the other examples.

1. A method of identifying an illumination light source in a surgical system, the method comprising steps of:
receiving a signal from a target after the target is illuminated by the illumination light source;
performing spectroscopic analysis of the received signal; and
based at least in part on the spectroscopic analysis, determining a characteristic of the illumination light source.

2. The method of claim 1, further comprising comparing the spectroscopic analysis to database information including spectroscopic information for different light types and/or different materials of the target, wherein the determination of the characteristic of the illumination light source is based at least in part on the comparing of the spectroscopic analysis.

3. The method of claim 2, wherein the spectroscopic analysis comprises comparing at least one of an intensity or a spectrum of the received signal to intensities or spectra associated with various types of illumination light sources from the target located within the database information.

4. The method of claim 2, wherein the target comprises a test target configured to reflect the illumination light without light absorption.

5. The method of claim 2, wherein the illumination light source comprises at least one of a Xenon light source and an LED light source.

6. The method of claim 2, further comprising retrieving database information from a server connected to the Internet.

7. The method of claim 6, wherein the database information comprises an artificial intelligence engine.

8. The method of claim 1, further comprising adjusting at least one of a parameter or a characteristic of the illumination light source to improve spectroscopic analysis of tissue based on the illumination light source identified.

9. The method of claim 8, wherein the parameter includes at least one of a brightness, power, wavelength and intensity and the characteristic includes a type of the illumination light source.

10. The method of claim 1, further comprising:
illuminating anatomic tissue with the illumination light;
performing spectroscopic analysis of a reflection of the illumination light; and
determining a characteristic of the anatomic tissue with the spectroscopic analysis.

11. The method of claim 10, wherein the characteristic includes one or more of a type, material, composition, a composition profile, a structure, and/or a hardness of the anatomic tissue.

12. The method of claim 10, further comprising:
adjusting at least one of a parameter or a characteristic of a treatment light source to improve treatment of the anatomic tissue based on the type of the anatomic tissue.

13. A method of treating a target comprising:
receiving a signal from the target after the target is illuminated by an illumination light source;
performing spectroscopic analysis of the received signal;
based at least in part on the spectroscopic analysis, determining a first characteristic of the illumination light source and a second characteristic of the target; and
based at least in part on the determined characteristics of the illumination light source and the target, operating a surgical system to treat the target.

14. The method of claim 13, wherein operating the surgical system comprises generating or adjusting one or more parameters including an operation mode, a power or energy, a pulse shape profile, an emission pulse regime and/or a combined output pulse train of the surgical system.

15. The method of claim 14, wherein adjusting the one or more parameters is automatically performed by a controller.

16. The method of claim 14, wherein adjusting the one or more parameters comprises prompting a user to accept suggested adjustments to the illumination light source.

17. The method of claim 14, wherein at least one of the spectroscopic analysis, the parameter of the illumination light source or the characteristic of the illumination light source are communicated via the Internet.

18. The method of claim 17, further comprising utilizing an artificial intelligence engine to generate the one or more parameters.

19. The method of claim 13, wherein receiving the signal from the target after the target is illuminated by the illumination light source comprises:
receiving a first signal from a test target after the test target is illuminated by the illumination light source; and
receiving a second signal from an anatomic target after the anatomic target is illuminated by the illumination light source.

20. The method of claim 19, wherein performing spectroscopic analysis of the received signal comprises:
performing spectroscopic analysis of the first signal to identify a type of light emitted by the illumination light source; and
performing spectroscopic analysis of the second signal to identify a characteristic of tissue of the anatomic target.

21. The method of claim 20, wherein the characteristic includes one or more of a type, material, composition, a composition profile, a structure, and/or a hardness of the tissue.

22. The method of claim 20, wherein:
the test target comprises a reflective surface; and
the illumination light source comprises a Xenon light source or an LED light source.
